(19) United States
(12) Patent Application Publication
(10) Pub. No.: US 2022/0287678 A1
(43) Pub. Date: Sep. 15, 2022

(54) ULTRASOUND IMAGING APPARATUS, ULTRASOUND IMAGING SYSTEM, METHOD OF OPERATING ULTRASOUND IMAGING APPARATUS, COMPUTER-READABLE RECORDING MEDIUM, AND ULTRASOUND ENDOSCOPE SYSTEM

(71) Applicant: OLYMPUS CORPORATION, Tokyo (JP)

(72) Inventor: Kei TAGUCHI, Tokyo (JP)

(73) Assignee: OLYMPUS CORPORATION, Tokyo (JP)

(21) Appl. No.: 17/831,611

(22) Filed: Jun. 3, 2022

Related U.S. Application Data
(63) Continuation of application No. PCT/JP2019/047971, filed on Dec. 6, 2019.

(51) Int. Cl. A61B 8/00; G06T 7/00

(52) U.S. Cl. CPC A61B 8/4254; G06T 7/0014; A61B 8/4494; A61B 8/469; G06T 2207/10132

(57) ABSTRACT

An ultrasound imaging apparatus includes: a processor configured to set a detection position for detecting a shear wave that is generated due to an ultrasound wave that is applied to an observation target by an ultrasound transducer of an ultrasound probe, calculate feature data between the ultrasound transducer and the detection position, set a threshold in accordance with the feature data, acquire contact pressure between the ultrasound probe and the observation target, and determine whether the contact pressure is equal to or smaller than the threshold.

### CROSS-REFERENCE TO RELATED APPLICATION

This application is a continuation of International Application No. PCT/JP2019/047971, filed on Dec. 6, 2019, the entire contents of which are incorporated herein by reference.

### BACKGROUND

### 1. Technical Field

The present disclosure relates to an ultrasound imaging apparatus, an ultrasound imaging system, a method of operating an ultrasound imaging apparatus, a computer-readable recording medium, and an ultrasound endoscope system.

### 2. Related Art

In the related art in the medical an ultrasound imaging apparatus that generates an ultrasound image based on an ultrasound signal that is obtained by causing an ultrasound transducer to transmit and receive ultrasound waves to and from a subject that is an observation target has been used.

As the ultrasound imaging apparatus, an apparatus that sets a region of interest (ROI) in an ultrasound image, causes shear waves to occur in the region of interest by transmitting push pulses, receives track pulses for detecting a propagation state of the shear waves, and measures elastic property in the region of interest with high accuracy is known (for example, see Japanese 1 Laid-open Patent Publication No. 2015-126955). This measurement method is called shear wave elastography. Further, in the shear wave elastography, to reduce attenuation of the ultrasound waves, the ultrasound waves may be transmitted and received by bringing the ultrasound transducer or a balloon that covers the ultrasound transducer into contact with the subject in some cases.

### SUMMARY

In some embodiments, an ultrasound imaging apparatus includes: a processor configured to set a detection position for detecting a shear wave that is generated due to an ultrasound wave that is applied to an observation target by an ultrasound transducer of an ultrasound probe, calculate feature data between the ultrasound transducer and the detection position, set a threshold in accordance with the feature data, acquire contact pressure between the ultrasound probe and the observation target, and determine whether the contact pressure is equal to or smaller than the threshold.

In some embodiments, an ultrasound imaging system includes: the ultrasound imaging apparatus; and a detector configured to detect the contact pressure.

In some embodiments, a method of operating an ultrasound imaging apparatus includes: setting a detection position for detecting a shear that is generated due to an ultrasound wave that is applied to an observation target by an ultrasound transducer of an ultrasound probe, calculating feature data between the ultrasound transducer and the detection position; setting a threshold in accordance with the feature data, acquiring contact pressure between the ultrasound probe and the observation target, and determining whether the contact pressure is equal to or smaller than the threshold.

In some embodiments, provided is a non-transitory computer readable recording medium with an executable program stored thereon. The program causes an ultrasound imaging apparatus to execute: setting a detection position for detecting a shear that is generated due to an ultrasound wave that is applied to an observation target by an ultrasound transducer of an ultrasound probe, calculating feature data between the ultrasound transducer and the detection position, setting a threshold in accordance with the feature data, acquiring contact pressure between the ultrasound probe and the observation target, and determining whether the contact pressure is equal to or smaller than the threshold.

In some embodiments, an ultrasound endoscope system includes: an ultrasound endoscope including an insertion portion configured to be inserted into a subject, an ultrasound transducer that is arranged on a distal end of the insertion portion, the ultrasound transducer being configured to transmit and receive an ultrasound wave, and a sensor that is arranged on the distal end of the insertion portion, the sensor being configured to detect contact pressure between the ultrasound endoscope and the subject; and an ultrasound imaging apparatus including a processor configured to set a detection position for detecting a shear that is generated due to an ultrasound wave that is applied to an observation target by the ultrasound, calculate feature data between the ultrasound transducer and the detection position, set a threshold in accordance with the feature data, acquire contact pressure between the ultrasound robe and the observation target, and determine whether the contact pressure is equal to or smaller than the threshold.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a configuration of an ultrasound imaging system including an ultrasound imaging apparatus according to one embodiment;

FIG. 2 is a flowchart illustrating an outline of a process performed by the ultrasound imaging apparatus according to one embodiment;

FIG. 3 is a diagram illustrating an example of an ultrasound image;

FIG. 4 is a diagram illustrating an example of a measurement result;

FIG. 5 is a diagram illustrating an example of the measurement result;

FIG. 6 is a diagram illustrating an example of an ultrasound image in a case where contact pressure exceeds a threshold;

FIG. 7 is a block diagram illustrating a configuration of an ultrasound imaging system including an ultrasound imaging apparatus according to a first modification of one embodiment;

FIG. 8 is a diagram illustrating a relationship between contact pressure and a distance;

FIG. 9 is a flowchart illustrating an outline of a process performed by the ultrasound imaging apparatus according to the first modification of one embodiment;

FIG. 10 is a block diagram illustrating a configuration of an ultrasound imaging system including an ultrasound imaging apparatus according to a second modification of one embodiment;

FIG. 11 is a diagram illustrating a relationship between contact pressure and a density;

FIG. 12 is a flowchart illustrating an outline of a process performed by the ultrasound imaging apparatus according to the second modification of one embodiment;

FIG. 13 is a block diagram illustrating a configuration of an ultrasound imaging system including an ultrasound imaging apparatus according to a third modification of one embodiment;

FIG. 14 is a diagram illustrating a relationship between contact pressure and an attenuation coefficient;

FIG. 15 is a flowchart illustrating an outline of a process performed by the ultrasound imaging apparatus according to the third modification of one embodiment;

FIG. 16 is a block diagram illustrating a configuration of an ultrasound imaging system including an ultrasound imaging apparatus according to a fourth modification of one embodiment;

FIG. 17 is a diagram illustrating a relationship among contact pressure, a distance, and a density; and

FIG. 18 is a flowchart illustrating an outline of a process performed by the ultrasound imaging apparatus according to the fourth modification of one embodiment.

### DETAILED DESCRIPTION

Embodiments of an ultrasound imaging apparatus, an ultrasound imaging system, and an ultrasound imaging method according to the present disclosure will be described below with reference to the drawings. The present disclosure is not limited by the embodiments below. The present disclosure is applicable to general ultrasound imaging apparatuses, general ultrasound imaging systems, and general ultrasound imaging methods capable of performing observation by shear wave elastography.

### Embodiment

### Configuration of Ultrasound Imaging System

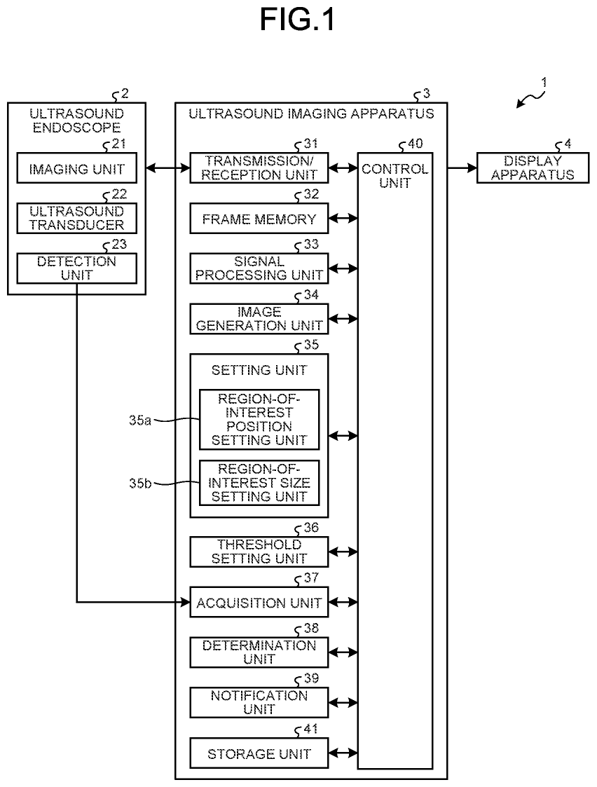

FIG. 1 is a block diagram illustrating a configuration of an ultrasound imaging system including an ultrasound imaging apparatus according to one embodiment. An ultrasound imaging system 1 includes an ultrasound endoscope 2 as an ultrasound probe, an ultrasound imaging apparatus 3, and a display apparatus 4. In the ultrasound imaging system 1, the ultrasound endoscope 2 and the ultrasound imaging apparatus 3 are connected to each other via a connector (not illustrated). Further, the display apparatus 4 displays an ultrasound image, tissue characteristic data that is obtained by analyzing the ultrasound image, or the like, and is connected to the ultrasound imaging apparatus 3.

The ultrasound endoscope 2 transmits ultrasound waves inside a body of a subject as an observation target, and receives ultrasound waves that are reflected by tissue inside the body of the subject. The ultrasound endoscope 2 includes, at a distal end of an insertion portion that is inserted into the subject, an imaging unit 21 that captures an image of the inside of the body of the subject, an ultrasound transducer 22 that transmits and receives ultrasound waves, and a detection unit 23 that detects contact pressure between the ultrasound endoscope 2 and the subject. However, the ultrasound probe not limited to the ultrasound endoscope, but may be an external ultrasound probe.

The imaging unit 21 includes an imaging optical system and an image sensor, is inserted into a digestive tract (an esophagus, a stomach, a duodenum, or a large intestine) or a respiratory organ (a trachea or a bronchus) of the subject, and is able to capture images of the digestive tract, the respiratory organ, or organs (a pancreas, a gallbladder, a bile duct, a biliary tract, lymph nodes, a mediastinal organ, a blood vessel, and the like) around the digestive tract or the respiratory organ. Further, the ultrasound endoscope 2 includes a light guide that guides illumination light that is applied to the subject at the time of imaging. A distal end portion of the light guide reaches the distal end of the insertion portion of the ultrasound endoscope 2 inserted into the subject, and a proximal end portion is connected to a light source device that generates the illumination light. Meanwhile, the ultrasound endoscope 2 may be configured without the imaging unit.

The ultrasound transducer 22 converts an electrical pulse signal received from the ultrasound imaging apparatus into an ultrasound pulse (acoustic pulse applies the ultrasound pulse to the subject, converts an ultrasound echo reflected by the subject into an electrical echo signal (ultrasound signal) that represents the ultrasound echo by a voltage change, and outputs the electrical echo signal. The ultrasound transducer 22 is of a convex type for example, but may be of a radial type or a linear type. Further, the ultrasound endoscope 2 may cause the ultrasound transducer 22 to mechanically perform scanning, or may include a plurality of piezoelectric elements that are arranged in an array as the ultrasound transducer 22 and cause the ultrasound transducer 22 to electronically perform scanning by electronically switching the piezoelectric elements related to transmission and reception or delaying transmission and reception performed by each of the piezoelectric elements. Furthermore, the ultrasound endoscope 2 may transmit and receive ultrasound waves while an outer periphery of the ultrasound transducer 22 is covered by a balloon, or may transmit and receive ultrasound waves by bringing the ultrasound transducer 22 in direct contact with the subject without using the balloon.

The detection unit 23 is, for example, a distortion sensor. The detection unit 23 outputs, as an electrical signal, an amount of distortion that is caused by pressure applied to the ultrasound endoscope 2.

The ultrasound imaging apparatus 3 transmits and receives an electrical signal to and from the ultrasound endoscope 2, and generates an ultrasound image by performing a predetermined process on the electrical signal received from the ultrasound endoscope 2. The ultrasound imaging apparatus 3 includes a transmission/reception unit 31, a frame memory 32, a signal processing unit 33, an image generation unit 34, a setting unit 35, a threshold setting unit 36, an acquisition unit 37, a determination unit 38, a notification unit 39, a control unit 40, and a storage unit 41.

The transmission/reception unit 31 transmits and receives an electrical signal to and from the ultrasound transducer 22. The transmission/reception unit 31 transmits a transmission drive wave signal with a predetermined waveform and at a predetermined transmission timing to the ultrasound transducer 22, and receives an electrical echo signal from the ultrasound transducer 22. Further, the transmission/reception unit 31 also has a function to transmit various control signals output from the control unit 40 to the ultrasound endoscope receive various kinds of information including an ID for identification from the ultrasound endoscope 2, and transmit the various kinds of information to the control unit 40.

The frame memory 32 is implemented by, for example, a ring buffer, and chronologically stores therein ultrasound images of a single frame generated by the image generation unit 34. The frame memory 32 may chronologically store therein ultrasound images of a plurality frames. In this case, if capacity f the frame memory 32 is insufficient (if ultrasound images of a predetermined number of frames are stored), the frame memory 32 overwrites the oldest ultrasound image with the latest ultrasound image to chronologically store latest ultrasound images of the predetermined number of frames.

The signal processing unit 33 generates digital reception data by using a signal received from the transmission/reception unit 31. The signal processing unit 33 performs processing, such as bandpass filtering, envelope detection, or logarithmic transformation, on the echo signal received by the transmission/reception unit 31, generates digital reception data for an ultrasound image, and outputs the reception data to the control unit 40. The signal processing unit 33 is implemented by a central processing unit (CPU) with arithmetic and control functions, various arithmetic circuits, or the like.

The image generation unit 34 generates data of various images including the ultrasound image by using information including the reception data generated by the signal processing unit 33. The image generation unit 34 generates a display image including the ultrasound image by using the reception data generated by the signal processing unit 33 and various kinds of predetermined data the image generation unit 34 is implemented by a CPU with arithmetic and control functions, various arithmetic circuits, or the like.

The setting unit 35 sets a detection position for detecting a propagation state of shear waves that are generated by applying ultrasound waves from the ultrasound transducer included in the ultrasound probe to the observation target. The setting unit 35 includes a region-of-interest position setting unit 35a and a region-of-interest size setting unit 35b. The region-of-interest position setting unit 35a sets a position of a region of interest (ROI), so that a detection position is set in the ROI. The region-of-interest size setting unit 35b sets a size of the ROI. The setting unit 35 is implemented by a CPU with arithmetic and control functions, various arithmetic circuits, or the like.

The threshold setting unit 36 sets a threshold. The threshold setting unit 36 sets, as the threshold, a value that is stored in the storage unit 41, for example. Further, the threshold setting unit 36 may set a different threshold in accordance with an organ to be observed. The threshold setting unit 36 is implemented by a CPU with arithmetic and control functions, various arithmetic circuits, or the like.

The acquisition unit 37 acquires contact pressure between the ultrasound endoscope 2 and the subject from the detection unit 23.

The determination unit 38 determines whether the contact pressure acquired by the acquisition unit 37 is equal to or smaller than the threshold that is set by the threshold setting unit 36. The determination unit 38 is implemented by a CPU with arithmetic and control functions, various arithmetic circuits, or the like.

The notification unit 39 gives a notice indicating that the contact pressure is equal to or smaller than the threshold on the basis of a determination result obtained by the determination unit 38. Specifically, the notification unit 39 gives a notice indicating that the contact pressure is equal to or smaller than the threshold by superimposing a predetermined mark or the like on the ultrasound image generated by the image generation unit 34. However, the notification unit 39 may give a notice indicating that the contact pressure is equal to or smaller than the threshold by sound or the like. The notification unit 39 is implemented by a CPU with arithmetic and control functions, various arithmetic circuits, or the like

The control unit 40 comprehensively controls entire operation of the ultrasound imaging system 1. The control unit 40 is implemented by a general-purpose processor, such as a CPU, with arithmetic and control functions, a dedicated integrated circuit, such as an application specific integrated circuit (ASIC) or a field programmable gate array (FPGA), that implements specific functions, or the like. If the control unit 40 is implemented by a general-purpose processor or an FPGA, the control unit 40 reads various programs and various kinds of data stored in the storage unit 41, and comprehensively controls the ultrasound imaging apparatus 3 by performing various kinds of arithmetic processing in relation to the operation of the ultrasound imaging apparatus 3. If the control unit 40 is implemented by an ASIC, it may be possible to individually perform various kinds of processing or may perform various kinds of processing by using various kinds of data stored in the storage unit 41. In the present embodiment, the control unit 40 and at least a part of the signal processing unit 33, the image generation unit 34, the setting unit 35, the threshold setting unit 36, the determination unit 38, and the notification unit 39 may be configured with a common general-purpose processor or a common dedicated integrated circuit. Further, the control unit 40 may have a function to execute shear wave elastography when the determination unit 38 determines that the contact pressure is equal to or smaller than the threshold. Meanwhile, execution of the shear wave elastography is to transmit push pulses from the ultrasound transducer 22 to the observation target to generate shear waves, and transmit and receive track pulses for detecting a propagation state of the shear waves between the ultrasound transducer 22 and the observation target.

The storage unit 41 stores therein various kinds of information needed for the operation of the ultrasound imaging apparatus 3. The storage unit 41 is configured with a read only memory (ROM) in which various programs or the like are pre-installed, a random access memory (RAM) for storing arithmetic parameters, data, or the like for each of processes, or the like.

It is possible to select the arithmetic parameters or the data for each of processes from a plurality or pieces of preset data stored in the ROM. Further, it is possible to automatically read a setting that was used in the past, on the basis of an ID for identifying a connected probe.

Furthermore, it is possible to cause the display apparatus 4 to display a setting associated with the ID for identification, in priority to other settings.

It is possible to store a calculation parameter or data that is used for each of processes and that is changed during an examination, as a data set as one of the pieces of preset data, together with a name of a related identifier or a related scope. The name of the related identifier or the related scope may be set automatically or may be set manually. Further, a user is allowed to arbitrarily change or add a name of the data set

As for the calculation parameter or the data that is used for each of processes and that is read as a preset, it may be possible to set and display only a part of the calculation parameter or the data included in information on an observation site, such as a digestive organ or a bronchus, which is a target for the connected probe, such that a user is able to edit the calculation parameter or the data on the basis or the information on the observation site.

If the scope connected to the ultrasound imaging apparatus is a scope that adopts, as a target, an observation site that is different from a target for a scope that was connected in the past, or if the scope connected to the ultrasound imaging apparatus is a scope that is connected to the ultrasound imaging apparatus tar the first time, scope information, such as an ID for identification, is added. Further, at this time, it is possible to newly add, as the information on the observation site, a name and a calculation parameter or data for each of processes. As for the added information on the observation site, it may be possible to set and display only a part of the calculation parameter or the data included in the information such that a user is able to edit the calculation parameter or the data when reading the preset data.

The display apparatus 4 is configured with liquid crystal, organic electro luminescence (EL), or the like, and displays an image including the ultrasound image generated by the image generation unit 34.

Ultrasound imaging method implemented by ultrasound imaging apparatus

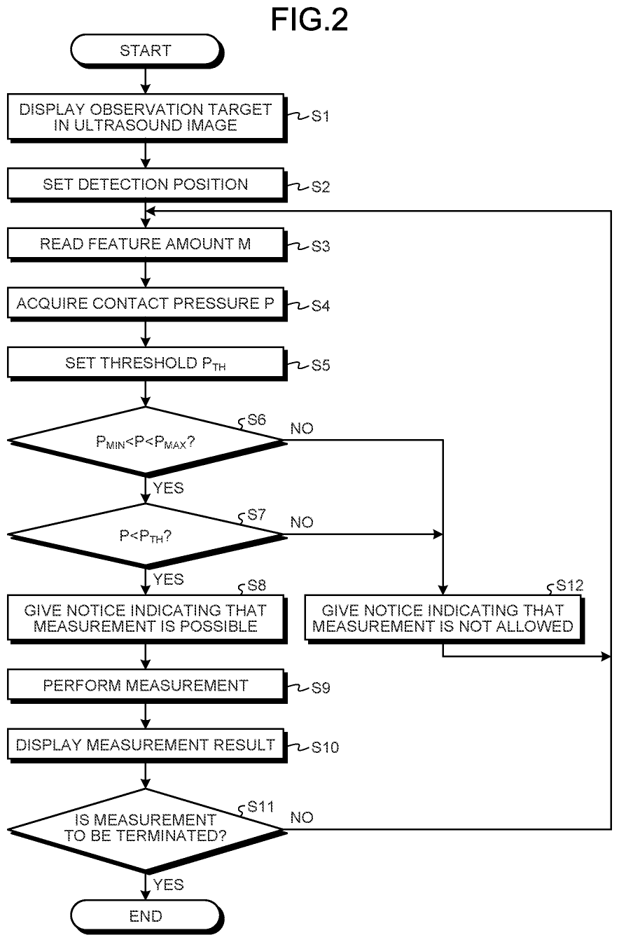

FIG. 2 is a flowchart illustrating an outline of a process performed by the ultrasound imaging apparatus according to one embodiment. First, an observation target is displayed in the ultrasound image by inputting operation from an input apparatus, such as a mouse (not illustrated) (Step S1).

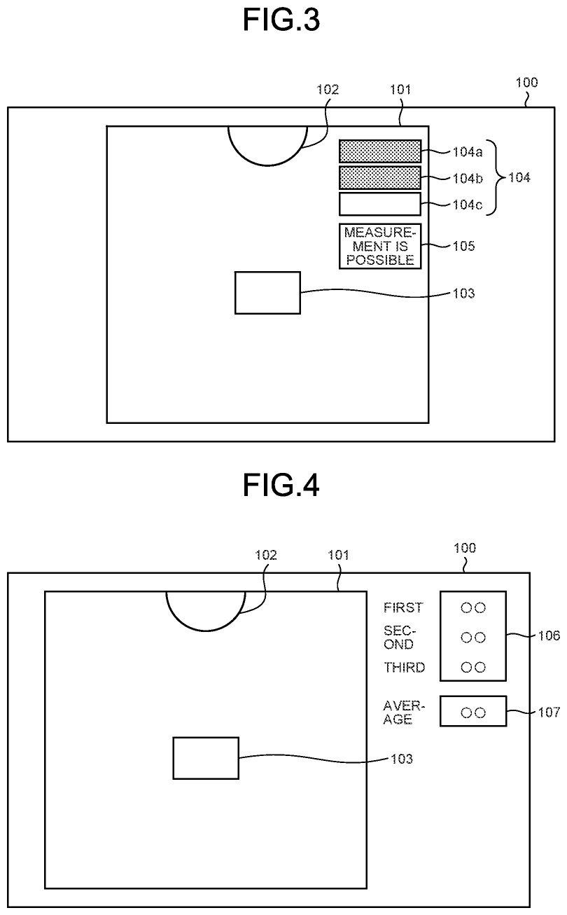

FIG. 3 is a diagram illustrating an example of the ultrasound image. Operation is input such that an observation target is located in a central part of an ultrasound image 101 that is displayed on a screen 100 of the display apparatus 4 as illustrated in FIG. 3. In an upper central part at the ultrasound image 101, a transducer area 102 corresponding to the ultrasound transducer 22 is located.

Subsequently, the setting unit 35 sets a detection position (Step S2). Specifically, the setting unit 35 sets an ROI in accordance with input of operation from the input apparatus, and sets the detection position in the ROI. The ROI set such that the observation target is included inside an ROI 103 that is located in the central part in FIG. 3, and sets the detection position in the ROI.

Thereafter, the control unit 40 reads feature data M (Step S3). The feature data M indicates an amount used to set a threshold for contact pressure. The control unit 40 may read, as the feature data M, an amount that is stored in advance in the storage unit 41, or may read, as the feature data M, an amount that is measured by the ultrasound endoscope 2 via the transmission/reception unit 31. Further, the control unit 40 may read, as the feature data M, an amount that is input by a user by using the input apparatus or an amount that is stored in a different server apparatus or the like that is connected via the Internet.

Subsequently, the acquisition unit 37 acquires contact pressure P between the ultrasound endoscope 2 and the subject from the detection unit 23 (Step S4).

Furthermore, the threshold setting unit 36 sets a threshold P_{TH} in accordance with the feature data M (Step S5).

Thereafter, the determination unit 38 determines whether the contact pressure P has a relationship such that P_{MIN}<P<P_{MAX} (Step S6). Here, P_{MIN} is a measurable lower limit value of the contact pressure P and P_{MAX} is a measureable upper limit value of the contact pressure P. If the contact pressure P deviates from the range of P_{MIN}<P<P_{MAX}, it is difficult to accurately perform measurement, so that it is preferable to perform measurement after adjusting the contact pressure P to an appropriate range. If the contact pressure P is too small, it is likely that the ultrasound endoscope 2 and the subject do not appropriately come into contact with each other, so that it may be difficult to accurately perform measurement. If the contact pressure P is, too large, tissue of the subject is compressed, so that it may be difficult to accurately perform measurement.

If the determination unit 38 determines that the contact pressure P has the relationship such that P_{MIN}<P<P_{MAX} (Step S6: Yes), the determination unit 38 determines whether the contact pressure P is equal to or smaller than the threshold P_{TH} (Step S7).

If the determination unit 38 determines that the contact pressure P is equal to or smaller than the threshold P_{TH} (Step S7: Yes), the notification unit 39 gives a notice indicating that measurement is possible (Step S8). Specifically, the notification unit 39 changes a color of a contact pressure display portion 104 to give a notice indicating that the measurement is possible. The color of the contact pressure display portion 104 is changed in order of contact pressure bars 104a, 104b, and 104c with an increase in the contact pressure P. FIG. 3 illustrates an example in which the colors of the contact pressure bars 104a and 104b are changed For example, if the contact pressure P is equal to or smaller than the threshold P_{TH}, the color of the contact pressure bar 104c is not changed, and the entire contact pressure display portion 104 is displayed in red. Further, the notification unit 39 may give a notice indicating that the measurement is possible by an con 105 that gives a notice indicating that the measurement is possible by characters. Furthermore, the notification unit 39 may give a notice indicating that the measurement is possible by changing a color of the ROI 103.

Subsequently, the ultrasound imaging apparatus 3 performs measurement (Step S9). The control unit 40 performs measurement in accordance with input of predetermined operation, for example. However, the control unit 40 may immediately perform the shear wave elastography the determination unit 38 determines that the contact pressure P is equal to or smaller than the threshold P_{TH}.

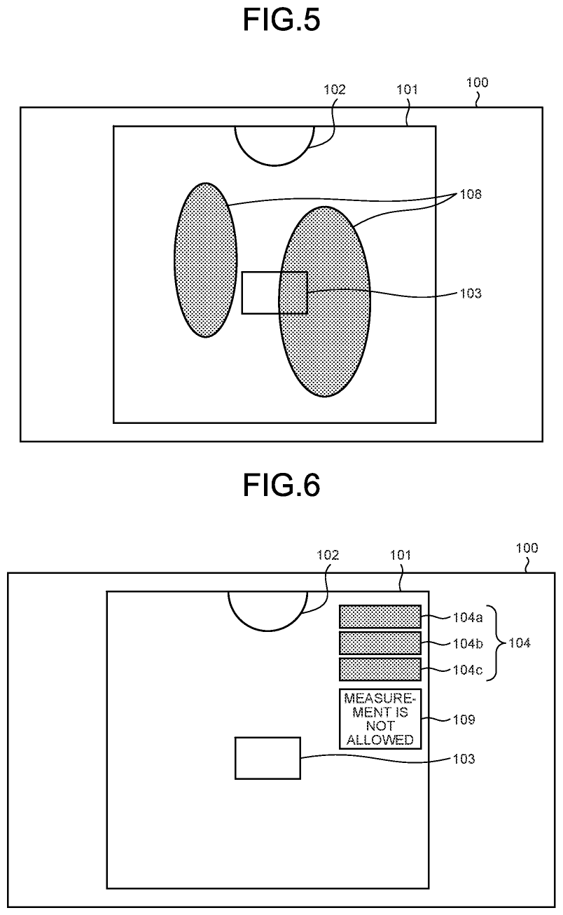

Thereafter, the ultrasound. imaging apparatus 3 causes the display apparatus 4 to display a measurement result (Step S10). FIG. 4 and FIG. 5 are diagrams illustrating examples of the measurement result. FIG. 4 illustrates measurement results 106 of measurement performed a plurality of number of times (three times in FIG. 4), and an average value 107 of all of the measurement results. The measurement results 106 and the average value 107 are numerical values corresponding to the contact pressure P. In this manner, the measurement results may be represented by numerical values. Further, in FIG. 5, color images 108 of shear waves representing the contact pressure P are displayed in a superimposed manner on the ultrasound image 101 on the basis of the measurement results. In this manner, the measurement results may be displayed by images.

Furthermore, the control unit 40 determines whether input indicating termination of measurement is received (Step S11), and if the control unit 40 determines that input indicating termination of measurement is received (Step S11: Yes), the control unit 40 terminates the series of processes.

At Step S6, if the determination unit 38 determines that the contact pressure P does not have the relationship such. that P_{MIN}<P<P_{MAX} (Step S6: No), the notification unit 39 gives a notice indicating that the measurement is not allowed (Step S12). Similarly, at Step S7, if the determination unit 38 determines that the contact pressure P is not equal to or smaller than the threshold P_{TH} (Step S7: No), the notification unit 39 gives a notice indicating that the measurement is not allowed (Step S12). FIG. 6 is a diagram illustrating an example of an ultrasound image in a case where the contact pressure exceeds the threshold. As illustrated in FIG. 6, if the contact pressure P exceeds the threshold P_{TH}, colors of all of the contact pressure bars 104a to 104c are changed. Alternatively, the not unit 39 may give a notice indicating that the measurement is allowed by an icon 109 that represents, by characters, that the measurement is allowed.

At Step S11, if the control unit 40 determines that input indicating termination of measurement is not received (Step S11: No), the process returns to Step S3 and the process is continued.

As described above, according to one embodiment, if the contact pressure P exceeds the threshold P_{TH}, measurement is not performed and a notice indicating that the measurement is not allowed is given, so that it is possible to perform measurement when the contact pressure P for an observation target is appropriate.

### First Modification

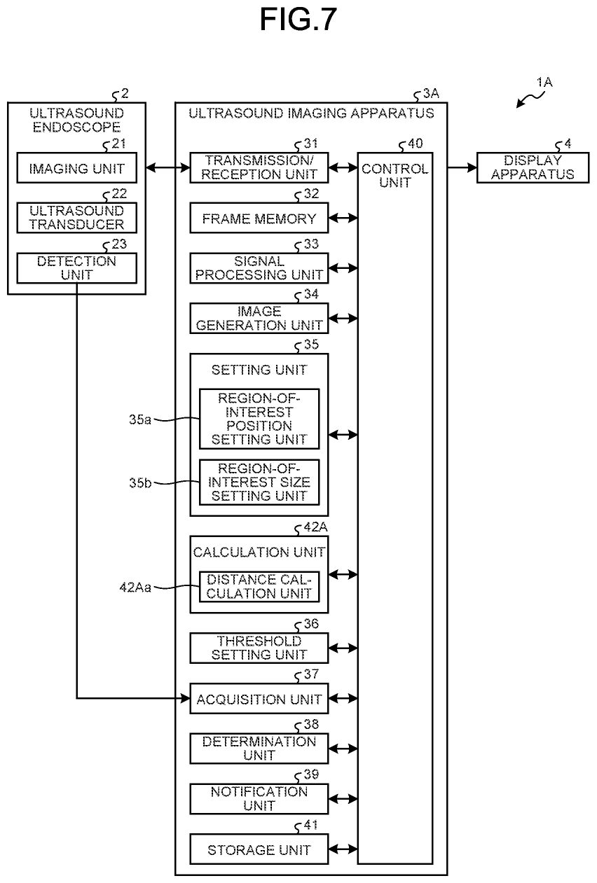

FIG. 7 is a block diagram illustrating a configuration of an ultrasound imaging system including an ultrasound imaging apparatus according to a first modification of one embodiment. An ultrasound imaging apparatus 3A of an ultrasound imaging system 1A according to the first modification of one embodiment includes a calculation unit 42A that calculates feature data between the ultrasound transducer 22 and the detection position. In the first modification, the feature data is a distance between the ultrasound transducer 22 and the detection position.

The calculation unit 42A includes a distance calculation unit 42Aa that calculates, as the feature data, the distance between the ultrasound transducer 22 and the detection position.

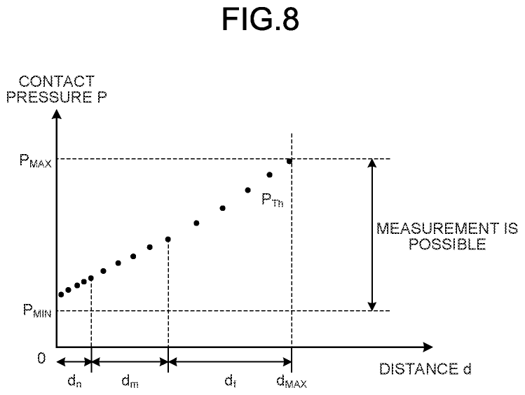

The threshold setting unit 36 sets a threshold in accordance with the feature data. The threshold setting unit 36 increases the threshold with an increase in the distance between the ultrasound transducer 22 and the detection position. FIG. 8 is a diagram illustrating a relationship between the contact pressure and the distance. Points illustrated in FIG. 8 represent thresholds P_{TH} at a plurality of distances d. When a region dₙ at a small distance d is to be measured, the contact pressure P has a large influence on the measurement result, and therefore, the threshold P_{TH} is set to a small value. In contrast, when a region d_{f} at a large distance d is to be measured, the contact pressure P has a small influence on the measurement result, and therefore, the threshold P_{TH} is set to a large value. In a region dₘ at an intermediate distance, the threshold P_{TH} is set to an intermediate value. A look-up table that is generated based on the relationship as illustrated in FIG. 8 is stored in the storage unit 41, and the threshold setting unit 36 reads a value corresponding to the feature data from the look-up table in the storage unit 41 and sets the read value as the threshold P_{TH}. Further, the threshold setting unit 36 may set the threshold P_{TH} from a different look-up table in accordance with an organ to be observed.

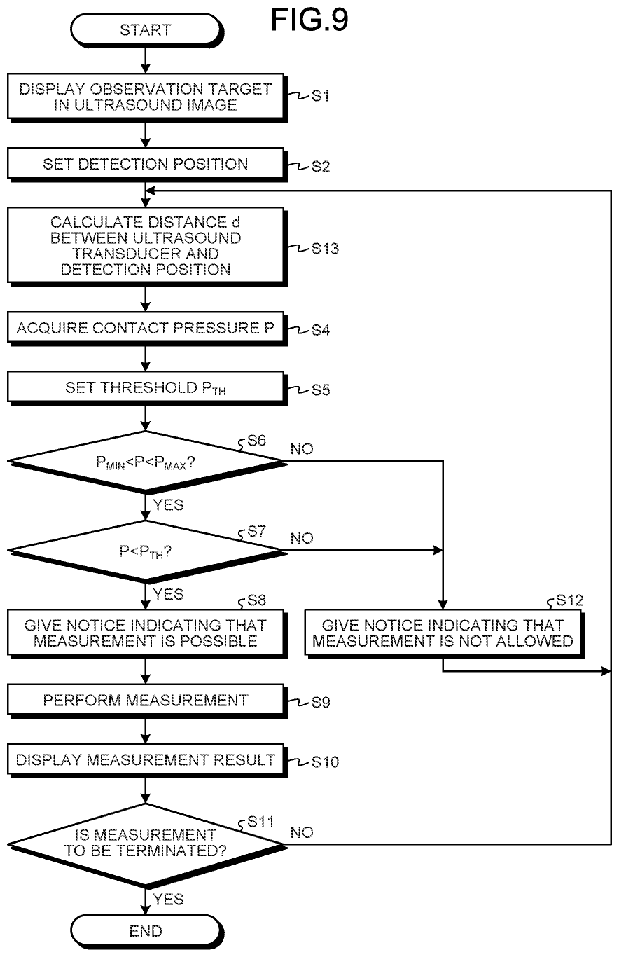

FIG. 9 is a flowchart illustrating an outline of a process performed by the ultrasound imaging apparatus according to the first modification of one embodiment. After Step S2, the distance calculation unit 42Aa calculates the distance d between the ultrasound transducer 22 and the detection position (Step S13).

Then, at Step S5, the threshold setting unit 36 increases the threshold P_{TH} with an increase in the distance d between the ultrasound transducer 22 and the detection position on the basis of the look-up table stored in the storage unit 41.

According to the first modification as described above, the threshold setting unit 36 increases the threshold P_{TH} with an increase in the distance d between the ultrasound transducer 22 and the detection position. The contact pressure P has a larger influence on a shallower portion of the observation target, where the shallower portion is located closer to the ultrasound transducer 22. Therefore, if a shallow portion in which the distance d between the ultrasound transducer 22 and the detection position is small is adopted as an observation target, the threshold setting unit 36 sets the threshold P_{TH} to a small value and prevents a situation in which measurement is not accurately performed due to the contact pressure P.

### Second Modification

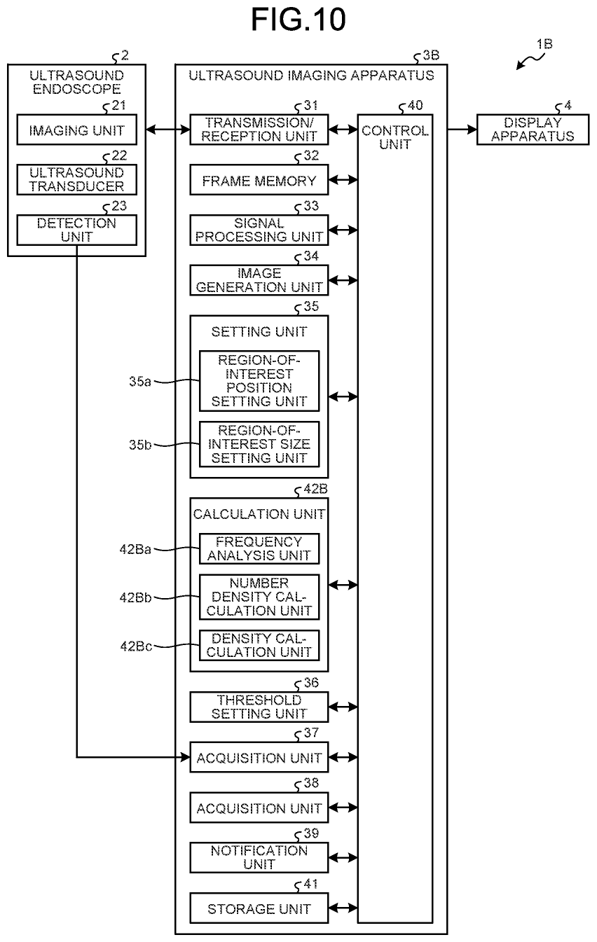

FIG. 10 is a block diagram illustrating a configuration of an ultrasound imaging system including an ultrasound imaging apparatus according to a second modification of one embodiment. An ultrasound imaging apparatus 3B of an ultrasound imaging system 1B according to the second modification of one embodiment includes a calculation unit 42B that calculates feature data between the ultrasound transducer 22 and the detection position. In the second modification, the feature data is a density of an observation target between the ultrasound transducer 22 and the detection position.

The calculation unit 42B includes a frequency analysis unit 42Ba that performs a frequency analysis on an echo signal acquired from the ultrasound transducer 22 and calculates a frequency spectrum, a number density calculation unit 42Bb that calculates a number density from the frequency spectrum, and a density calculation unit 42Bc that calculates a density from the number density.

The frequency analysis unit 42Ba repeatedly performs sampling of RF data (line data) of each of sound rays of the ultrasound transducer 22, where the RF data is generated by the transmission/reception unit 31, at predetermined time intervals, and generates sample data. The frequency analysis unit 42Ba performs a fast Fourier transform (FFT) process on a sample data group and calculates a frequency spectrum at a number of positions (data positions) on the RF data. The “frequency spectrum”described herein indicates a “frequency distribution with intensity at a certain reception depth”that is obtained by performing the FFT process on the sample data group. Further, the “intensity”described herein indicates, for example, any of a parameter, such as voltage of an echo signal, electric power of the echo signal, sound pressure of an ultrasound echo, or acoustic energy of the ultrasound echo, amplitude of the parameter, a time integral value of the parameter, and a combination of the above-described values.

In general, if the observation target is living tissue, the frequency spectrum of the echo signal tends to differ depending on characteristics of the living tissue that is scanned by ultrasound waves. This is because the frequency spectrum is correlated with a size, a number density, acoustic impedance, or the like of a scattering body that causes the ultrasound waves to scatter. The “characteristics of the living tissue”indicates, for example, a malignant tumor (cancer), a benign tumor, an endocrine tumor, a mucinous tumor, normal tissue, a cyst, or a vascular.

The number density calculation unit 42Bb approximates the frequency spectrum calculated by the frequency analysis unit 42Ba by a first-degree equation, and calculates feature data (a slope, an intercept, or a center frequency) that characterizes the first-degree equation. Further, the number density calculation unit 42Bb calculates a number density by comparing the calculated feature data with feature data of a plurality of reference scattering bodies for which number densities or the like are already known.

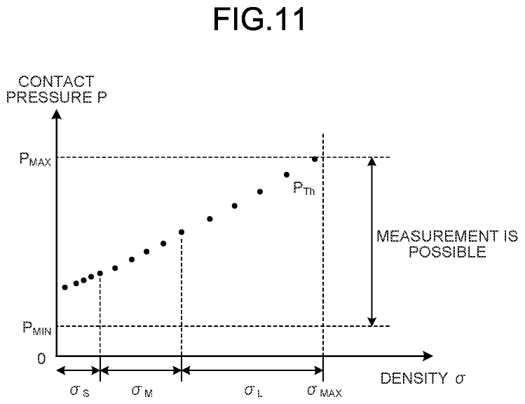

The threshold setting unit 36 sets a threshold in accordance with the feature data. The threshold setting unit 36 increases the threshold with an increase in the density of the observation target between the ultrasound transducer 22 and the detection position. FIG. 11 is a diagram. illustrating a relationship between the contact pressure and the density. Points illustrated in FIG. 11 represent thresholds P_{TH} at a plurality of densities σ. When a region σ_{S} with a small density σ is to be measured, the contact pressure P has a lame influence on a measurement result, and therefore, the threshold P_{TH} is set to a small value. In contrast, when a region σ_{L} with a large density σ is to be measured, the contact pressure P has a small influence on the measurement result, and therefore, the threshold P_{TH} is set to a large value. In a region σ_{M} with an intermediate density σ, the threshold P_{TH} is set to an intermediate value. A look-up table that is generated based on the relationship as illustrated in FIG. 11 is stored in the storage unit 41, and the threshold setting unit 36 reads a value corresponding to the feature data from. the look-up table in the storage unit 41 and sets the read value as the threshold P_{TH}. Further, the threshold setting unit 36 may set the threshold P_{TH} from a different look-up table in accordance with an organ to be observed.

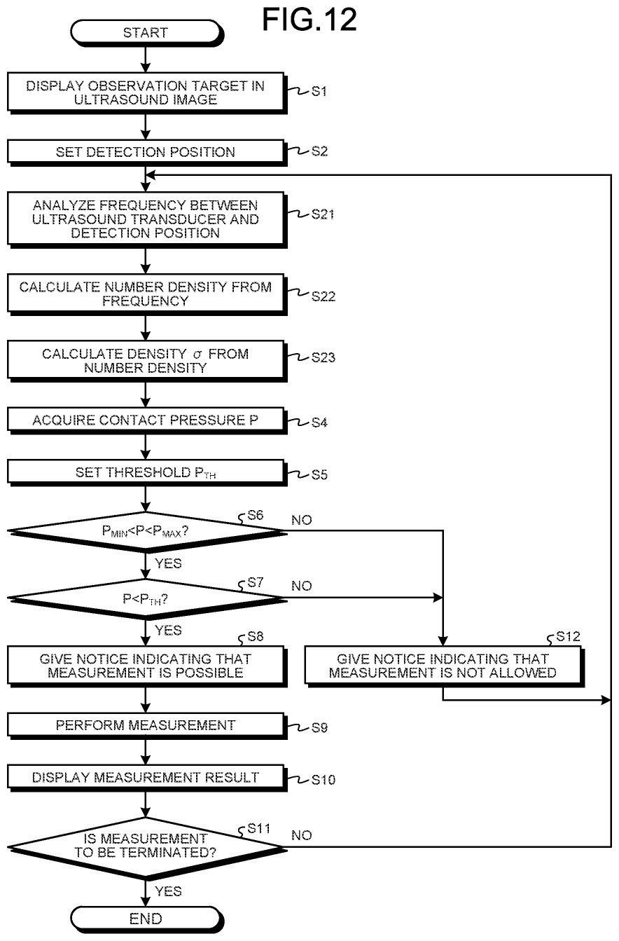

FIG. 12 is a flowchart illustrating an outline of a process performed by the ultrasound imaging apparatus according to the second modification of one embodiment, After Step S2, the frequency analysis unit 42Ba performs a frequency analysis on the echo signal acquired from the ultrasound transducer 22 and calculates a frequency spectrum (Step S21).

Subsequently, the number density calculation unit 42Bb calculates a number density from the frequency spectrum (Step S22).

Furthermore, the density calculation unit 42Bc calculates the density o from the number density (Step S23).

Then, at Step S1, the threshold setting unit 36 increases the threshold P_{TH} with an increase in the density σ between the ultrasound transducer 22 and the detection position on the basis of the look-up table stored in the storage unit 41.

According to the second modification of one embodiment as described above, the threshold se tine unit 36 increases the threshold P_{TH} with an increase in the density σ between the ultrasound transducer 22 and the detection position. The observation target has a larger influence on the contact pressure P with a decrease in the density σ of the observation target. Therefore, if the density between the ultrasound transducer 22 and the detection position is small, the threshold setting unit 36 sets the threshold P_{TH} to a small value and prevents a situation in which measurement is not accurately performed due to the contact pressure P.

### Third Modification

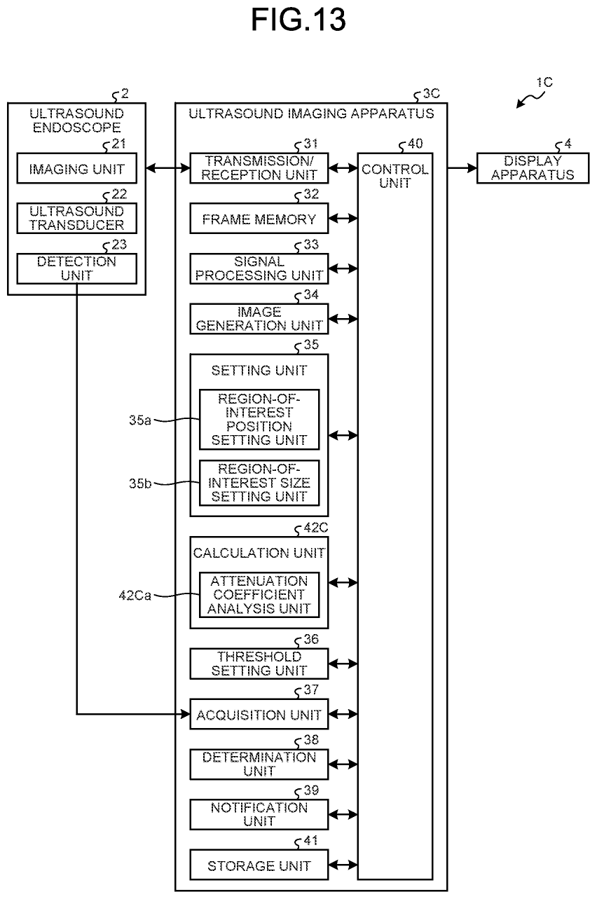

FIG. 13 is a block diagram illustrating a configuration of an ultrasound imaging system including, an ultrasound imaging apparatus according to a third modification of one embodiment. An ultrasound imaging apparatus 3C of an ultrasound imaging system 1C according to the third modification of one embodiment includes a calculation unit 42C that calculates feature data between the ultrasound transducer 22 and the detection position. In the third modification, the feature data is an attenuation coefficient between the ultrasound transducer 22 and the detection position.

The calculation unit 42C includes an attenuation coefficient analysis unit 42Ca that analyzes an attenuation coefficient on the basis of the echo signal acquired from the ultrasound transducer 22.

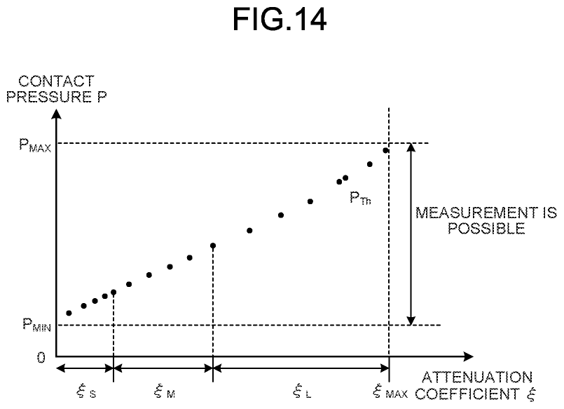

The threshold setting unit 36 sets a threshold in accordance with the feature data. The threshold setting unit 36 increases the threshold with an increase in the attenuation coefficient between the ultrasound transducer 22 and the detection position. FIG. 14 is a diagram illustrating a relationship between the contact pressure and. the attenuation coefficient. Points illustrated in FIG. 14 represent the thresholds P_{TH} at a plurality of attenuation coefficients ξ. When a region in which the attenuation coefficient ξ is small is to be measured, the contact pressure P has a large influence on the measurement result, and therefore, the threshold P_{TH} is set to a small value. In contrast, when a region ξ_{L} in which the attenuation coefficient ξ is large is to be measured, the contact pressure P has a small influence on the measurement result, and therefore, the threshold P_{TH} is set to a large value. In a region ξ_{M} in which the attenuation coefficient ξ is intermediate, the threshold P_{TH} is set to an intermediate value. A look-up table that is generated based on the relationship as illustrated in FIG. 14 is stored in the storage unit 41, and the threshold setting unit 36 reads a value corresponding to the feature data from the look-up table in the storage unit 41 and sets the read value as the feature data. Further, the threshold setting unit 36 may set the threshold P_{TH} from a different look-up table in accordance with an organ to be observed.

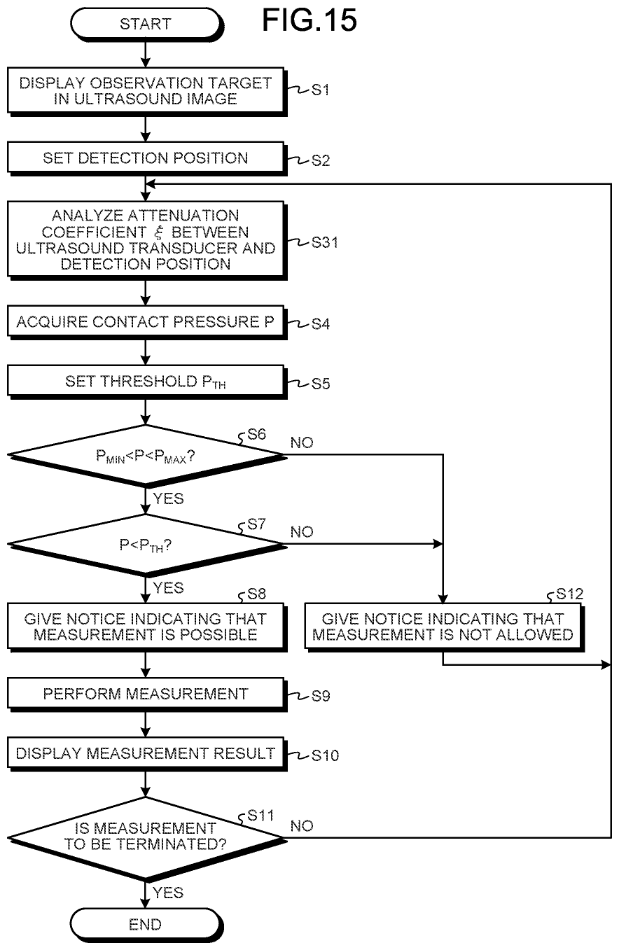

FIG. 15 is a flowchart illustrating an outline of a process performed by the ultrasound imaging apparatus according to the fourth modification of one embodiment. After Step S2, the attenuation coefficient analysis unit 42Ca analyzes the attenuation coefficient between the ultrasound transducer 22 and the detection position (Step S31).

Further, at Step S5, the threshold setting unit 36 increases the threshold P_{TH} with an increase in the attenuation coefficient ξ between the ultrasound transducer 22 and the detection position on the basis of the look-up table stored in the storage un it 41.

According to the third modification as described above, the threshold setting unit 36 increases the threshold P_{TH} with an increase in the attenuation coefficient ξ between the ultrasound transducer 22 and the detection position. The threshold setting unit 36 sets the threshold P_{TH} to a small value if the attenuation coefficient ξ between the ultrasound transducer 22 and the detection position is small, and prevents a situation in which measurement is not accurately performed due to the contact pressure P.

### Fourth Modification

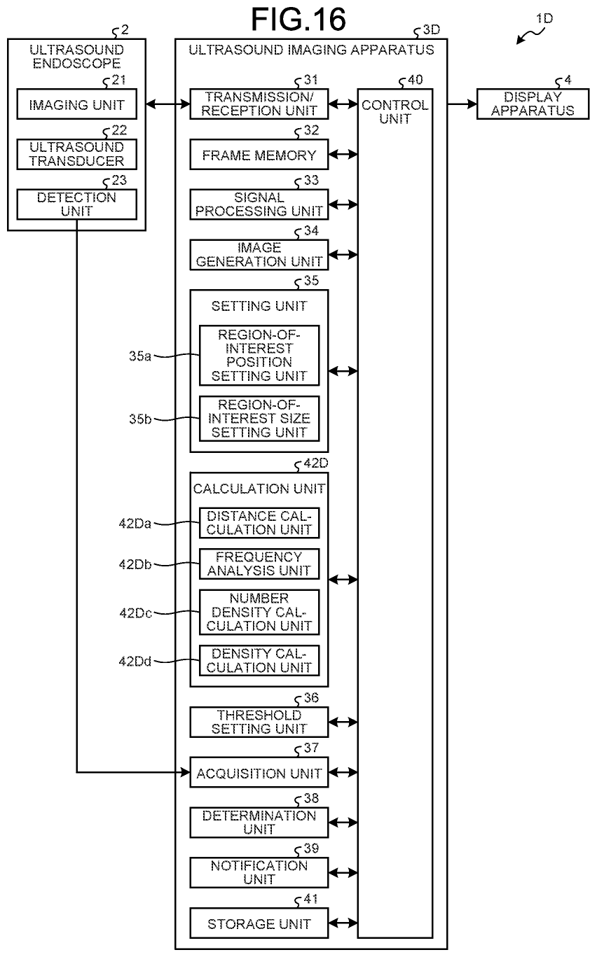

FIG. 16 is a block diagram illustrating a configuration of an ultrasound imaging system of an ultrasound imaging apparatus according to a fourth modification of one embodiment. An ultrasound imaging apparatus 3D of an ultrasound imaging system 1D according to the fourth modification of one embodiment includes a calculation unit 42D that calculate feature data between the ultrasound transducer 22 and the detection position. In the fourth modification, the feature data is a distance between the ultrasound transducer 22 and the detection position and a density of an observation target between the ultrasound transducer 22 and the detection position.

The calculation unit 42D includes a distance calculation unit 42Da that calculates, as the feature data, a distance between the ultrasound transducer 22 and the detection position, a frequency analysis unit 42Db that performs a frequency analysis on the echo signal acquired from the ultrasound transducer 22 and calculates a frequency spectrum, a number density calculation unit 42Dc that calculates a number density from the frequency spectrum, and a density calculation unit 42Dd that calculates a density from the number density.

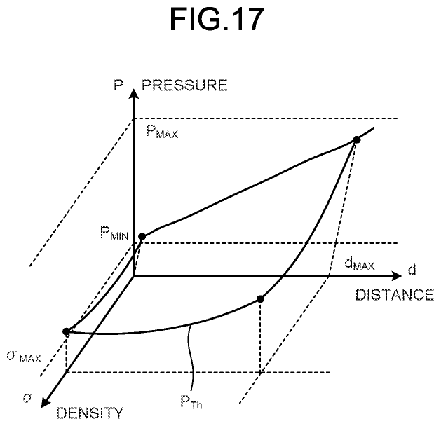

The threshold setting unit 36 sets a threshold in accordance with the feature data. The threshold setting unit 36 increases the threshold with a increase in the distance between the ultrasound transducer 22 and the detection position, and increases the threshold with an increase in the density of the observation target between the ultrasound transducer 22 and the detection positon. FIG. 17 is a diagram illustrating a relationship among the contact pressure, the distance, and the density. Points illustrated in FIG. 17 represent the thresholds P_{TH} at each of the distances d and each of the densities σ. When a region in which the distance d and the density σ are small is to be measured, the contact pressure P has a large influence on the measurement result, and therefore, the threshold P_{TH} is set to a small value contrast, when a region in which the distance and the density σ are large is to be measured, the contact pressure P has a small influence on the measurement result, and therefore, the threshold P_{TH} is set to a large value. A look-up table that is generated based on the relationship as illustrated in FIG. 17 stored in the storage unit 41, and the threshold setting unit 36 reads value corresponds to the feature data from the look-up table in the storage unit 41 and sets the read value as the threshold P_{TH}. Further, the threshold setting unit 36 may set threshold P_{TH} from a different look-up table in accordance with an organ to be observed.

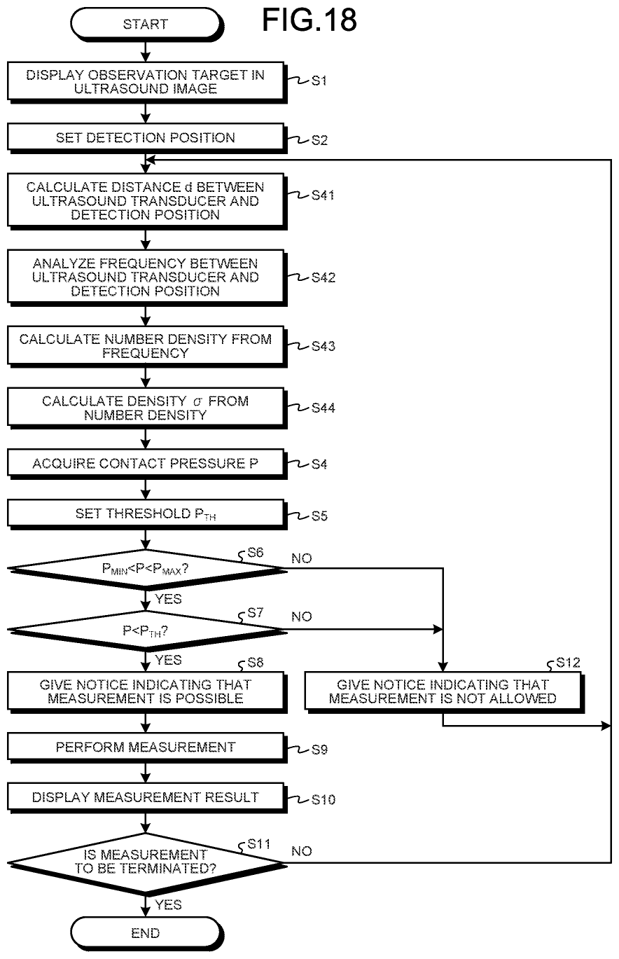

FIG. 18 is a flowchart illustrating an outline of a process performed oy the ultrasound imaging apparatus according to the fourth modification of one embodiment. After Step S2, the distance calculation unit 42Da calculates the distance d between the ultrasound transducer 22 and the detection position (Step S41).

The frequency anal is unit 42Db performs a frequency analysis on the echo signal acquired from the ultrasound transducer 22 and calculates a frequency spectrum (Step S42).

Subsequently, the number density calculation unit 42Dc calculates a number density from the frequency spectrum (Step S43).

Furthermore, the density calculation unit 42Dd calculates the density σ from the number density (Step S44).

Moreover, at Step S5, the threshold setting unit 36 increases the threshold P_{TH} with an increase in the distance d between the ultrasound transducer 22 and the detection position and increases the threshold P_{TH} with an increase in the density of the observation target between the ultrasound transducer 22 and the detection position on the basis of the look-up table stored in the storage unit 41.

According to the fourth modification of one embodiment as described above, the threshold setting unit 36 increases the threshold P_{TH} with an increase in the distance d between the ultrasound transducer 22 and the detection position and increases the threshold P_{TH} with an increase in the density o of the observation target between the ultrasound transducer 22 and the detection position The threshold setting unit 36 sets the threshold P_{TH} to a small value if the distance d and the density σ between the ultrasound transducer 22 and the detection position are small, and prevents a situation in which measurement is not accurately performed due to the contact pressure P.

According to one embodiment of the present disclosure, it is possible to implement an ultrasound imaging apparatus, an ultrasound imaging system, a method of operating an ultrasound imaging apparatus, a computer-readable recording medium, and an ultrasound endoscope system capable of performing measurement when contact pressure is appropriate for an observation target.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound imaging apparatus comprising:
a processor configured to
set a detection position for detecting a shear wave that is generated due to an ultrasound wave that is applied to an observation target by an ultrasound transducer of an ultrasound probe,
calculate feature data between the ultrasound transducer and the detection position,
set a threshold in accordance with the feature data,
acquire contact pressure between the ultrasound probe and the observation target, and
determine whether the contact pressure is equal to or smaller than the threshold.

2. The ultrasound imaging apparatus according to claim 1, wherein the feature data is a distance between the ultrasound transducer and the detection position.

3. The ultrasound imaging apparatus according to claim 2, wherein the processor is configured to set the threshold such that the threshold increases with an increase in the distance between the ultrasound transducer and the detection position.

4. The ultrasound imaging apparatus according to claim 1, wherein the feature data is a density of the observation target between the ultrasound transducer and the detection position.

5. The ultrasound imaging apparatus according to claim 4, wherein the processor is configured to set the threshold such that the threshold increases with an increase in the density of the observation target between the ultrasound transducer and the detection position.

6. The ultrasound imaging apparatus according to claim 1, wherein the feature data is an attenuation coefficient between the ultrasound transducer and the detection position.

7. The ultrasound imaging apparatus according to claim 6, wherein the processor is configured to set the threshold such that the threshold increases with an increase in the attenuation coefficient between the ultrasound transducer and the detection position.

8. The ultrasound imaging apparatus according to claim 1, wherein the feature data is a distance between the ultrasound transducer and the detection position, and a density of the observation target between the ultrasound transducer and the detection position.

9. The ultrasound imaging apparatus according to claim 8, wherein the processor is configured to set the threshold such that the threshold increases with an increase in the distance between the ultrasound transducer and the detection position, and such that the threshold increases with an increase in the density of the observation target between the ultrasound transducer and the detection position.

10. The ultrasound imaging apparatus according to claim 1, wherein if the contact pressure is equal to or smaller than the threshold, the processor is configured to perform shear wave elastography.

11. The ultrasound imaging apparatus according to claim 1, wherein the processor is configured to give a notice indicating that the contact pressure is equal to or smaller than the threshold.

12. The ultrasound imaging apparatus according to claim 1, wherein the contact pressure is an electrical signal that is based on pressure between the ultrasound probe and the observation target, the pressure being detected by the ultrasound probe.

13. The ultrasound imaging apparatus according claim 1, wherein the processor is configured to set the threshold in accordance with an organ including the observation target.

14. The ultrasound imaging apparatus according to claim 1, wherein the processor is configured to acquire an echo signal based on an ultrasound wave received by the ultrasound transducer, and calculate the feature data based on the echo signal.

15. The ultrasound imaging apparatus according 14, wherein the processor is configured to calculate a frequency spectrum from the echo signal, and calculate the feature data based on the frequency spectrum.

16. The ultrasound imaging apparatus according to claim 1, wherein the processor configured to determine whether the contact pressure is equal to or larger than a second threshold.

17. An ultrasound imaging system comprising:
the ultrasound imaging apparatus according to claim 1; and
a detector configured to detect the contact pressure.

18. A method of operating an ultrasound imaging apparatus comprising:
setting a detection position for detecting a shear that is generated due to an ultrasound wave that is applied to an observation target by an ultrasound transducer of an ultrasound probe,
calculating feature data between the ultrasound transducer and the detection position;
setting a threshold in accordance with the feature data,
acquiring contact pressure between the ultrasound probe and the observation target, and
determining whether the contact pressure is equal to or smaller than the threshold.

19. A non-transitory computer readable recording medium with an executable program stored thereon, the program causing an ultrasound imaging apparatus to execute:
setting a detection position for detecting a shear that is generated due to an ultrasound wave that is applied to an observation target by an ultrasound transducer of an ultrasound probe,
calculating feature data between the ultrasound transducer and the detection position,
setting a threshold in accordance with the feature data,
acquiring contact pressure between the ultrasound probe and the observation target, and
determining whether the contact pressure is equal to or smaller than the threshold.

20. An ultrasound endoscope system comprising:
an ultrasound endoscope including
an insertion portion configured to be inserted into a subject,
an ultrasound transducer that is arranged on a distal end of the insertion portion, the ultrasound transducer being configured to transmit and receive an ultrasound wave, and
a sensor that is arranged on the distal end of the insertion portion, the sensor being configured to detect contact pressure between the ultrasound endoscope and the subject; and
an ultrasound imaging apparatus including
a processor configured to
set a detection position for detecting a shear that is generated due to an ultrasound wave that is applied to an observation target by the ultrasound,
calculate feature data between the ultrasound transducer and the detection position,
set a threshold in accordance with the feature data,
acquire contact pressure between the ultrasound probe and the observation target, and
determine whether the contact pressure is equal to or smaller than the threshold.
